SEQUENCE LISTING (148 sequences)

SEQ ID NO: 1 - DNA, 33 bp, Homo sapiens
tcggtcagaggctagctacaacgatgcgttgct

SEQ ID NO: 2 - DNA, 33 bp, Homo sapiens
ggcgtacgaggctagctacaacgactgctcggt

SEQ ID NO: 3 - DNA, 33 bp, Homo sapiens
ggcggcgtaggctagctacaacgagacctgctc

SEQ ID NO: 4 - DNA, 33 bp, Homo sapiens
ctcgggtcaggctagctacaacgactgggtagc

SEQ ID NO: 5 - DNA, 33 bp, Homo sapiens
tcctctgcaggctagctacaacgacggggtcct

SEQ ID NO: 6 - DNA, 33 bp, Homo sapiens
actctgcaaggctagctacaacgatctgcgagc

SEQ ID NO: 7 - DNA, 33 bp, Homo sapiens
gggcgacgaggctagctacaacgatctgcaatt

SEQ ID NO: 8 - DNA, 33 bp, Homo sapiens
aaggggcgaggctagctacaacgagactctgca

SEQ ID NO: 9 - DNA, 33 bp, Homo sapiens
aaaacgggaggctagctacaacgacaggttgta

SEQ ID NO: 10 - DNA, 33 bp, Homo sapiens
agaataaaaggctagctacaacgagggaccagg

SEQ ID NO: 11 - DNA, 33 bp, Homo sapiens
atggcagaaggctagctacaacgaaaaacggga

SEQ ID NO: 12 - DNA, 33 bp, Homo sapiens
aactgggtaggctagctacaacgaggcagaata

SEQ ID NO: 13 - DNA, 33 bp, Homo sapiens
atccaaaaaggctagctacaacgatgggtatgg

SEQ ID NO: 14 - DNA, 33 bp, Homo sapiens
aggggaagaggctagctacaacgaaaaaatcca

SEQ ID NO: 15 - DNA, 33 bp, Homo sapiens
ttttaaaaaggctagctacaacgatatcttgga

SEQ ID NO: 16 - DNA, 33 bp, Homo sapiens
gtggggggaggctagctacaacgagggaaggct

SEQ ID NO: 17 - DNA, 33 bp, Homo sapiens
gttgaatgaggctagctacaacgattgctttcg

SEQ ID NO: 18 - DNA, 33 bp, Homo sapiens
gtcgttgaaggctagctacaacgagatttgctt

SEQ ID NO: 19 - DNA, 33 bp, Homo sapiens
ggcccggaaggctagctacaacgaccgcgcgcg

SEQ ID NO: 20 - DNA, 33 bp, Homo sapiens
tcacctccaggctagctacaacgaggcctcggc

SEQ ID NO: 21 - DNA, 33 bp, Homo sapiens
ccgccgtcaggctagctacaacgactccatggc

SEQ ID NO: 22 - DNA, 33 bp, Homo sapiens
ggtggctcaggctagctacaacgaccagcgcgg

SEQ ID NO: 23 - DNA, 33 bp, Homo sapiens
cgttgagcaggctagctacaacgaggcggggtg

SEQ ID NO: 24 - DNA, 33 bp, Homo sapiens
ccgcgtccaggctagctacaacgagtaggagtg

SEQ ID NO: 25 - DNA, 33 bp, Homo sapiens
cagcgggtaggctagctacaacgatgcgccgcg

SEQ ID NO: 26 - DNA, 33 bp, Homo sapiens
gcacatccaggctagctacaacgactcctccgg

SEQ ID NO: 27 - DNA, 33 bp, Homo sapiens
aaaagcacaggctagctacaacgaccacctcct

SEQ ID NO: 28 - DNA, 33 bp, Homo sapiens
taaaaagcaggctagctacaacgaatccacctc

SEQ ID NO: 29 - DNA, 33 bp, Homo sapiens
gaccgtcgaggctagctacaacgagttaaaaag

SEQ ID NO: 30 - DNA, 33 bp, Homo sapiens
ttgccttgaggctagctacaacgacgtcgatgt

SEQ ID NO: 31 - DNA, 33 bp, Homo sapiens
agggcgggaggctagctacaacgagtggttgcc

SEQ ID NO: 32 - DNA, 33 bp, Homo sapiens
tggccctgaggctagctacaacgacgagtttcc

SEQ ID NO: 33 - DNA, 33 bp, Homo sapiens
acctctgcaggctagctacaacgacgtggccct

SEQ ID NO: 34 - DNA, 33 bp, Homo sapiens
cggagggtaggctagctacaacgactctgcacc

SEQ ID NO: 35 - DNA, 33 bp, Homo sapiens
ggcggcacaggctagctacaacgactggctccc

SEQ ID NO: 36 - DNA, 33 bp, Homo sapiens
cgggcggcaggctagctacaacgaacctggctc

SEQ ID NO: 37 - DNA, 33 bp, Homo sapiens
agggatccaggctagctacaacgagaagcagag

SEQ ID NO: 38 - DNA, 33 bp, Homo sapiens
gggtagggaggctagctacaacgaccatgaagc

SEQ ID NO: 39 - DNA, 33 bp, Homo sapiens
gggctgagaggctagctacaacgatccaggggg

SEQ ID NO: 40 - DNA, 33 bp, Homo sapiens
gtggatggaggctagctacaacgagtcttggag

SEQ ID NO: 41 - DNA, 33 bp, Homo sapiens
cgtggtggaggctagctacaacgaggacgtctt

SEQ ID NO: 42 - DNA, 33 bp, Homo sapiens
gggggtagaggctagctacaacgaggagagggg

SEQ ID NO: 43 - DNA, 33 bp, Homo sapiens
ggaggaggaggctagctacaacgagaggccggg

SEQ ID NO: 44 - DNA, 33 bp, Homo sapiens
gccccccgaggctagctacaacgaaaggaggag

SEQ ID NO: 45 - DNA, 33 bp, Homo sapiens
ccggggagaggctagctacaacgagtccttcgg

SEQ ID NO: 46 - DNA, 33 bp, Homo sapiens
ggacagcgaggctagctacaacgagggtccggg

SEQ ID NO: 47 - DNA, 33 bp, Homo sapiens
tggggtggaggctagctacaacgaagcgatggg

SEQ ID NO: 48 - DNA, 33 bp, Homo sapiens
cttgaggcaggctagctacaacgatctttctcg

SEQ ID NO: 49 - DNA, 33 bp, Homo sapiens
cacctggtaggctagctacaacgattgaggcac

SEQ ID NO: 50 - DNA, 33 bp, Homo sapiens
gcaggggcaggctagctacaacgactggtactt

SEQ ID NO: 51 - DNA, 33 bp, Homo sapiens
ccagcttcaggctagctacaacgagctgtcggg

SEQ ID NO: 52 - DNA, 33 bp, Homo sapiens
gtgggacgaggctagctacaacgatccagcttc

SEQ ID NO: 53 - DNA, 33 bp, Homo sapiens
ggagtgggaggctagctacaacgagactccagc

SEQ ID NO: 54 - DNA, 33 bp, Homo sapiens
atgctgccaggctagctacaacgagggagtggg

SEQ ID NO: 55 - DNA, 33 bp, Homo sapiens
gggcggtcaggctagctacaacgagctgccacg

SEQ ID NO: 56 - DNA, 33 bp, Homo sapiens
gaggctccaggctagctacaacgaccagggcgg

SEQ ID NO: 57 - DNA, 33 bp, Homo sapiens
gtgggtcgaggctagctacaacgagaggaggct

SEQ ID NO: 58 - DNA, 33 bp, Homo sapiens
aggtggtgaggctagctacaacgaggggtggtg

SEQ ID NO: 59 - DNA, 33 bp, Homo sapiens
actcgggcaggctagctacaacgagtagggcgg

SEQ ID NO: 60 - DNA, 33 bp, Homo sapiens
ggagctgtaggctagctacaacgatcgggcacg

SEQ ID NO: 61 - DNA, 33 bp, Homo sapiens
ggacttgcaggctagctacaacgaccgaagccg

SEQ ID NO: 62 - DNA, 33 bp, Homo sapiens
gggcctggaggctagctacaacgattgcatccg

SEQ ID NO: 63 - DNA, 33 bp, Homo sapiens
tgtgctggaggctagctacaacgacgggccttg

SEQ ID NO: 64 - DNA, 33 bp, Homo sapiens
gttcacacaggctagctacaacgatccctgcct

SEQ ID NO: 65 - DNA, 33 bp, Homo sapiens
cagttcacaggctagctacaacgaactccctgc

SEQ ID NO: 66 - DNA, 33 bp, Homo sapiens
cacagttcaggctagctacaacgaacactccct

SEQ ID NO: 67 - DNA, 33 bp, Homo sapiens
gttgccccaggctagctacaacgaagttcacac

SEQ ID NO: 68 - DNA, 33 bp, Homo sapiens
tcgccgccaggctagctacaacgaagtggggtc

SEQ ID NO: 69 - DNA, 33 bp, Homo sapiens
cccgtgccaggctagctacaacgactcgccgcc

SEQ ID NO: 70 - DNA, 33 bp, Homo sapiens
ggcgttgcaggctagctacaacgaaggtagtgt

SEQ ID NO: 71 - DNA, 33 bp, Homo sapiens
tggcttctaggctagctacaacgagccctcgtc

SEQ ID NO: 72 - DNA, 33 bp, Homo sapiens
gggctctgaggctagctacaacgagcctggctt

SEQ ID NO: 73 - DNA, 33 bp, Homo sapiens
gggaccccaggctagctacaacgacggagcccg

SEQ ID NO: 74 - DNA, 33 bp, Homo sapiens
ggtgggggaggctagctacaacgacccaccgga

SEQ ID NO: 75 - DNA, 33 bp, Homo sapiens
ggcgggggaggctagctacaacgaccgagggcc

SEQ ID NO: 76 - DNA, 33 bp, Homo sapiens
gggctgggaggctagctacaacgagggcaggga

SEQ ID NO: 77 - DNA, 33 bp, Homo sapiens
cgtcgaggaggctagctacaacgaccgcccctc

SEQ ID NO: 78 - DNA, 33 bp, Homo sapiens
gggctggcaggctagctacaacgacttcccgta

SEQ ID NO: 79 - DNA, 33 bp, Homo sapiens
cgatgcccaggctagctacaacgaccggggcgg

SEQ ID NO: 80 - DNA, 33 bp, Homo sapiens
gctccacgaggctagctacaacgagcccatccg

SEQ ID NO: 81 - DNA, 33 bp, Homo sapiens
ccggctccaggctagctacaacgagatgcccat

SEQ ID NO: 82 - DNA, 33 bp, Homo sapiens
tctccgcaaggctagctacaacgaccggctcca

SEQ ID NO: 83 - DNA, 33 bp, Homo sapiens
ccgtcagcaggctagctacaacgagtctccgca

SEQ ID NO: 84 - DNA, 33 bp, Homo sapiens
tccccggcaggctagctacaacgacggctcggt

SEQ ID NO: 85 - DNA, 33 bp, Homo sapiens
cccccgcgaggctagctacaacgagctcgtccg

SEQ ID NO: 86 - DNA, 33 bp, Homo sapiens
gtagggagaggctagctacaacgacccaggctg

SEQ ID NO: 87 - DNA, 33 bp, Homo sapiens
gggcgggcaggctagctacaacgacaaggcgcc

SEQ ID NO: 88 - DNA, 33 bp, Homo sapiens
cgggaaggaggctagctacaacgatcgcccgcg

SEQ ID NO: 89 - DNA, 33 bp, Homo sapiens
tagtcctcaggctagctacaacgagcggccccg

SEQ ID NO: 90 - DNA, 33 bp, Homo sapiens
tccccgacaggctagctacaacgactccagtcc

SEQ ID NO: 91 - DNA, 33 bp, Homo sapiens
tttccccgaggctagctacaacgaacctccagt

SEQ ID NO: 92 - DNA, 33 bp, Homo sapiens
tgagcgcgaggctagctacaacgacctcagttt

SEQ ID NO: 93 - DNA, 33 bp, Homo sapiens
ggaccacaaggctagctacaacgaaggtggttg

SEQ ID NO: 94 - DNA, 33 bp, Homo sapiens
cttggaccaggctagctacaacgaaacaggtgg

SEQ ID NO: 95 - DNA, 33 bp, Homo sapiens
aaacttggaggctagctacaacgacacaacagg

SEQ ID NO: 96 - DNA, 33 bp, Homo sapiens
ctgattaaaggctagctacaacgattggaccac

SEQ ID NO: 97 - DNA, 33 bp, Homo sapiens
tggtgctgaggctagctacaacgataaacttgg

SEQ ID NO: 98 - DNA, 33 bp, Homo sapiens
tgatgatcaggctagctacaacgactctgtctg

SEQ ID NO: 99 - DNA, 33 bp, Homo sapiens
tggtgatgaggctagctacaacgacatctctgt

SEQ ID NO: 100 - DNA, 33 bp, Homo sapiens
gcttggtgaggctagctacaacgagatcatctc

SEQ ID NO: 101 - DNA, 33 bp, Homo sapiens
atgggaacaggctagctacaacgaccgccgtcc

SEQ ID NO: 102 - DNA, 33 bp, Homo sapiens
gaatgggaaggctagctacaacgaatccgccgt

SEQ ID NO: 103 - DNA, 33 bp, Homo sapiens
tgacaggaaggctagctacaacgagggaacatc

SEQ ID NO: 104 - DNA, 33 bp, Homo sapiens
agtaaatgaggctagctacaacgaaggaatggg

SEQ ID NO: 105 - DNA, 33 bp, Homo sapiens
cacagtaaaggctagctacaacgagacaggaat

SEQ ID NO: 106 - DNA, 33 bp, Homo sapiens
gcccggccaggctagctacaacgaagtaaatga

SEQ ID NO: 107 - DNA, 33 bp, Homo sapiens
ccacaaacaggctagctacaacgacctgtagtg

SEQ ID NO: 108 - DNA, 33 bp, Homo sapiens
gtccacaaaggctagctacaacgaatcctgtag

SEQ ID NO: 109 - DNA, 33 bp, Homo sapiens
ccacgtccaggctagctacaacgaaaacatcct

SEQ ID NO: 110 - DNA, 33 bp, Homo sapiens
ccaagaccaggctagctacaacgagtccacaaa

SEQ ID NO: 111 - DNA, 33 bp, Homo sapiens
ccaccaagaggctagctacaacgacacgtccac

SEQ ID NO: 112 - DNA, 33 bp, Homo sapiens
gctggtccaggctagctacaacgacaagaccac

SEQ ID NO: 113 - DNA, 33 bp, Homo sapiens
gctctggtaggctagctacaacgacgccagtgg

SEQ ID NO: 114 - DNA, 33 bp, Homo sapiens
ctgcacccaggctagctacaacgattgccgctc

SEQ ID NO: 115 - DNA, 33 bp, Homo sapiens
cacactgcaggctagctacaacgaccacttgcc

SEQ ID NO: 116 - DNA, 33 bp, Homo sapiens
ctttccacaggctagctacaacgatgcacccac

SEQ ID NO: 117 - DNA, 33 bp, Homo sapiens
gcctttccaggctagctacaacgaactgcaccc

SEQ ID NO: 118 - DNA, 33 bp, Homo sapiens
ttcctggcaggctagctacaacgagctgccctc

SEQ ID NO: 119 - DNA, 33 bp, Homo sapiens
gtggacgtaggctagctacaacgaaggcggttt

SEQ ID NO: 120 - DNA, 33 bp, Homo sapiens
ccgggtggaggctagctacaacgagtacaggcg

SEQ ID NO: 121 - DNA, 33 bp, Homo sapiens
cctggcgcaggctagctacaacgaccagtgcgc

SEQ ID NO: 122 - DNA, 33 bp, Homo sapiens
caaatgaaaggctagctacaacgattcctggcg

SEQ ID NO: 123 - DNA, 33 bp, Homo sapiens
tttcccaaaggctagctacaacgagaaacttcc

SEQ ID NO: 124 - DNA, 33 bp, Homo sapiens
attgttggaggctagctacaacgagcccccttg

SEQ ID NO: 125 - DNA, 33 bp, Homo sapiens
tgggtcacaggctagctacaacgatgttggacg

SEQ ID NO: 126 - DNA, 33 bp, Homo sapiens
tctgggtcaggctagctacaacgaattgttgga

SEQ ID NO: 127 - DNA, 33 bp, Homo sapiens
gcacaatcaggctagctacaacgactgggtcac

SEQ ID NO: 128 - DNA, 33 bp, Homo sapiens
ggagcacaaggctagctacaacgacatctgggt

SEQ ID NO: 129 - DNA, 33 bp, Homo sapiens
actggagcaggctagctacaacgaaatcatctg

SEQ ID NO: 130 - DNA, 33 bp, Homo sapiens
atggagggaggctagctacaacgatggagcaca

SEQ ID NO: 131 - DNA, 33 bp, Homo sapiens
tggtacttaggctagctacaacgaggagggact

SEQ ID NO: 132 - DNA, 33 bp, Homo sapiens
gggctggtaggctagctacaacgattatggagg

SEQ ID NO: 133 - DNA, 33 bp, Homo sapiens
tcaacgataggctagctacaacgagcagccggg

SEQ ID NO: 134 - DNA, 33 bp, Homo sapiens
cctcaacgaggctagctacaacgaatgcagccg

SEQ ID NO: 135 - DNA, 33 bp, Homo sapiens
tcacctcaaggctagctacaacgagatatgcag

SEQ ID NO: 136 - DNA, 33 bp, Homo sapiens
cgtcgttcaggctagctacaacgactcaacgat

SEQ ID NO: 137 - DNA, 33 bp, Homo sapiens
gtaaagataggctagctacaacgagcgtgttgg

SEQ ID NO: 138 - DNA, 33 bp, Homo sapiens
aagtaaagaggctagctacaacgaatgcgtgtt

SEQ ID NO: 139 - DNA, 33 bp, Homo sapiens
ggcaatgaaggctagctacaacgatgggtttct

SEQ ID NO: 140 - DNA, 33 bp, Homo sapiens
tcacggcaaggctagctacaacgagaactgggt

SEQ ID NO: 141 - DNA, 33 bp, Homo sapiens
aggcagtcaggctagctacaacgaggcaatgaa

SEQ ID NO: 142 - DNA, 33 bp, Homo sapiens
atctcggcaggctagctacaacgatctggtagg

SEQ ID NO: 143 - DNA, 33 bp, Homo sapiens
gctgagtaaggctagctacaacgactcggcatt

SEQ ID NO: 144 - DNA, 33 bp, Homo sapiens
tattatcaaggctagctacaacgatttcagctg

SEQ ID NO: 145 - DNA, 33 bp, Homo sapiens
gggttattaggctagctacaacgacaattttca

SEQ ID NO: 146 - DNA, 33 bp, Homo sapiens
aaggggttaggctagctacaacgatatcaattt

SEQ ID NO: 147 - DNA, 33 bp, Homo sapiens
ctcccggaaggctagctacaacgacctttggca

SEQ ID NO: 148 - DNA, 33 bp, Homo sapiens
gtacatggaggctagctacaacgatcaaagttc

(19) United States
(12) Patent Application Publication
(10) Pub. No.: US 2021/0161939 A1
(43) Pub. Date: Jun. 3, 2021

(54) COMPOSITION FOR TREATING A PATIENT WITH A RESPIRATORY DISEASE CAUSED BY CHRONIC INFLAMMATION, PRODUCTION METHOD, AND USE OF SAID COMPOSITION

(71) Applicant: STERNA BIOLOGICALS GMBH & CO. KG, Marburg (DE)

(72) Inventor: Jonas Renz, München (DE)

(73) Assignee: STERNA BIOLOGICALS GMBH & CO. KG, Marburg (DE)

(21) Appl. No.: 16/955,806

(22) PCT Filed: Dec. 20, 2018
(86) PCT No.: PCT/EP2018/086438

§ 371 Date: Jun. 19, 2020

(30) Foreign Application Priority Data
Dec. 22, 2017 (EP) 17210504.1

(51) Int. Cl. A61K 31/711; A61K 9/00; A61K 45/06

(52) U.S. Cl. CPC A61K 31/711; A61K 45/06; A61K 9/008

(57) ABSTRACT

The invention relates to a composition for treating a patient suffering from a respiratory tract disease associated with chronic inflammations, the composition comprising at least one DNAzyme which specifically downregulates the expression of GATA-3 and/or comprising at least one DNAzyme which specifically downregulates the expression of Tbet. The composition is characterized in that the concentration of the DNAzyme in the composition is lower than 75 mg/ml. The invention also relates to a method for producing the composition according to the invention and to the use of the composition drug for treating a patient suffering from a respiratory tract disease associated with chronic inflammations.

The invention relates to a composition for treating a patient suffering from a respiratory tract disease associated with chronic inflammations as claimed in claim 1, to a method for producing the composition as claimed in claim 10 and to the use of such a composition as claimed in claim 12.

Unless otherwise indicated, the terms “expression” and “gene expression” are used synonymously below.

Chronic inflammations are of increasing significance in medicine. Accordingly, it is important to find therapies for diseases such as, for example, respiratory tract diseases associated with chronic inflammations. Such respiratory tract diseases are, for example, type I hypersensitivities, asthma or chronic obstructive pulmonary diseases (COPD).

The genesis of chronic inflammatory reactions and of autoimmune diseases (which are often also inflammation-dependent) involves, inter alia, two transcription factors: the Th1 cell-specific transcription factor Tbet and the Th2 cell-specific transcription factor GATA-3. In this connection, Tbet induces the specific development of Th1 cells; by contrast, GATA-3 induces the specific development of Th2 cells. The balance between Th1 cells and Th2 cells is thus shifted in favor of the Th2 cells by the expression of GATA-3 and/or by the inhibition of Tbet. Analogously, the inhibition of GATA-3 and/or the expression of Tbet ensures a rising Th1 cell level. Numerous scientific publications have been published in relation to the specific equilibrium between GATA-3 and Tbet or between Th1 cells and Th2 cells.

Consequently, attempts have been made in recent years to specifically inhibit or “switch off” the transcription factors GATA-3 and Tbet. One way of accomplishing this is the inhibition of the gene expression of these two transcription factors, modern gene technology providing various “tools” for this purpose. One of these tools is DNAzymes.

DNAzymes represent a comparatively new class of antisense molecules. Antisense molecules refer to molecules which are able to bind single-stranded nucleic acids (generally mRNA) in a specific and substantially complementary manner. At the same time, a peculiarity of DNAzymes is that they exhibit not only this binding function, but also a catalytic function. They are thus capable of specifically cleaving single-stranded target DNA or target RNA and of thus degrading them (Sel et al. 2008). In this case, reference is also made to post-transcriptional inhibition because the inhibition of gene expression takes place at the mRNA level, i.e. even before the translation of the mRNA to form a protein can follow.

Usually, DNAzymes of the 10-23 type are used for this purpose (Sontoro et al., 1997). Such DNAzymes have a catalytic domain of 15 nucleotides which is flanked by two substrate-binding domains. Said catalytic domain can comprise especially the conserved sequence ggctagctacaacga (SEQ ID No. 154). The specified sequence ggctagctacaacga is merely a preferred embodiment. A person skilled in the art is aware that DNAzymes of the 10-23 type having a modified catalytic domain can have a comparable biological activity. By contrast, the length of the substrate-binding domains is variable, it also being possible for two corresponding substrate-binding domains to differ from one another. In a preferred embodiment, the length of the substrate-binding domains is between 6 and 14 nucleotides, particular preference being given to a length of 9 nucleotides. Such DNAzymes have a specific sequence such as, for example, nnnnnnnnnggctagctacaacgannnnnnnnn. Generally, the substrate-binding domains are completely complementary to the regions flanking the intended cleavage site of the target mRNA—to bind and cleave the target RNA, it is, however, not absolutely necessary for the DNAzyme to be completely complementary. DNAzymes of the 10-23 type cleave the target mRNA at sequences containing purine and pyrimidine in sequence.

DNAzymes are just as known for their use for specifically inhibiting the expression of GATA-3 or Tbet. For example, WO 2005/033314 A2 describes the mode of action of various DNAzymes. The disclosure content of WO 2005/033314 is considered to be the technological background of the present invention.

Problems which can occur when using DNAzymes as active ingredient are often attributable to the comparatively high instability and the sensitivity of nucleic acids. If they are not present in physiologically favorable solutions, nucleic acids tend to degrade rapidly, for instance by enzymatic degradation or physical stress—this applies especially to single-stranded nucleic acids, including DNAzymes as well.

Furthermore, with any use of an active ingredient for therapeutic purposes, there follows the question of the administrability of the active ingredient. For instance, especially in the area of respiratory tract diseases associated with inflammations, a local administration is necessary when seeking to control the center of inflammation efficiently and to reach the target cells in a specific manner. Accordingly, in the case of type I hypersensitivities, asthma, or COPD for instance, an active ingredient is most effective when it can be taken up via the lungs.

One way of controlling respiratory tract diseases in a specific manner is, for example, inhalation. By means of inhalation, patients can take up active ingredients into the lungs or via the target cells in the lungs, the result being that the active ingredient can act in a site-specific manner or be taken up rapidly into the bloodstream via the alveoli. This method can reduce the risks of adverse effects in the rest of the organism. In addition, a lower dose is required because a substantially larger proportion of the dose reaches the site of action than by means of nonspecific administration.

Treatment strategies for patients with inflammatory diseases that are standard practice are limited especially to active ingredients which have a highly immunosuppressant action. Examples of such active ingredients are corticosteroids, mesalazine (5-aminosalicylic acid), ciclosporin, tacrolimus or TNHα antibodies. Nevertheless, none of the aforementioned therapeutic methods is suitable for being used in a specific manner. On the contrary, the substances used are known for their highly immunosuppressant action. Furthermore, the substances are associated with commonly occurring adverse effects, such as, for example, immunosuppression, hepatotoxicity, myocardial hypertrophy, gastrointestinal complaints, fatigue and dizziness, diarrhea, emesis, nephrotoxicity, neurotoxicity, tremor, hypertension, insomnia, depressions, cramps, masking of infections, hyperkalemia, hyperglycemia, increased risk of tumors—to name but just a few.

It is therefore the object of the invention to provide a DNAzyme-containing composition for treating a patient suffering from a respiratory tract disease associated with chronic inflammations that is suitable for specific administration. In particular, it is intended that the composition be able to be taken up via the lungs of the patient to be treated, and so said composition should be easy to atomize. Furthermore, it is intended that the active ingredient minimize the risk of adverse effects.

According to the invention, this object is achieved by a composition for treating a patient suffering from a respiratory tract disease associated with chronic inflammations, the composition comprising at least one DNAzyme which specifically downregulates the expression of GATA-3 and/or comprising at least one DNAzyme which specifically downregulates the expression of Tbet. In this connection, the composition is characterized in that the DNAzyme is present in the composition in a concentration lower than 75 mg/ml. What can be provided in particular is that the composition is administrable as an aerosol which is taken up via the lungs. Preferably, the composition can be a liquid or a solution; alternatively, it can be a powder.

A person skilled in the art is aware that downregulation of expression can be understood to mean either a partial inhibition or a complete inhibition of the expression. In any case, the expression is significantly reduced in comparison with the natural expression level. In the present case, the specific DNAzymes bind and cleave in vivo the mRNA of the transcription factors GATA-3 and/or Tbet, the proteins of which are central key molecules for the genesis of Th1- and Th2-dependent chronic inflammatory diseases. As a result of this mRNA cleavage, the mRNAs can no longer be translated into functional proteins. Consequently, the GATA-3 and/or Tbet protein level is significantly minimized.

The composition according to the invention having a DNAzyme concentration lower than 75 mg/ml can, then, be ideally used for treating patients suffering from a respiratory tract disease associated with chronic inflammation. This is especially because compositions having DNAzyme concentrations exceeding 75 mg/ml are associated with a loss of active ingredient. The reason therefor is that, owing to their increased viscosity, such high-concentration compositions can no longer be atomized in a problem-free manner (for instance, via an inhaler). Manual inhalers that are standard in medicine generally operate with an atomization pressure of from 40 bar to 45 bar. In the case of such an inhaler, the surface tension of a solution having a DNAzyme concentration of over 75 mg/ml is too high in order to still be atomizable. As a consequence, compositions having a DNAzyme concentration of 75 mg/ml already show a loss of active ingredient of about 6% when they are administered by means of an inhaler. In contrast, especially compositions having a DNAzyme concentration of from 20 mg/ml to 50 mg/ml have an optimal action effect in the case of treatment using an inhaler because compositions having such concentrations can be easily atomized. Accordingly, what is preferred according to the invention is a composition which has a DNAzyme concentration of from 20 mg/ml to 50 mg/ml.

The composition according to the invention can, then, be present especially as an inhalation solution which can be administered as an aerosol into the lungs of a patient, and this is advantageous especially in the treatment of respiratory tract diseases triggered by allergic inflammatory reactions. In the case of such respiratory tract diseases, the active ingredients (DNAzymes) can be administered essentially at the site of inflammation by a composition according to the invention in aerosol form. The result is a higher efficiency in the treatment of corresponding patients. For this purpose, what is provided in particular is that the composition is a liquid or a solution. Alternatively, the composition can be a powder.

The feature according to the invention that the DNAzyme concentration is lower than 75 mg/ml is, then, highly advantageous especially when using DNAzymes. In the case of aqueous DNA solutions, concentrations which far exceed 75 mg/ml are usually possible without the viscosity of the solution being significantly increased at the same time. For instance, especially relatively small DNA molecules (<1000 bp), which also include DNAzymes, can be present in concentrations of over 100 mg/ml without the solutions becoming particularly viscous at the same time.

However, what becomes apparent in the case of DNAzymes is the surprising effect of DNAzyme-containing solutions already becoming disproportionately highly viscous at concentrations of at least 75 mg/ml. At concentrations of over 75 mg/ml, they even precipitate. The reason therefor is especially the three-dimensional structure of the DNAzymes, which is characterized by the formation of a “loop”. Owing to this particular structure, DNAzymes can—promoted by their single-strandedness—form a kind of polymer structure and easily clump together, and this in turn results in an altogether poor solubility of high amounts of DNAzyme. For the site-specific treatment of patients suffering from a respiratory tract disease associated with chronic inflammation, the composition according to the invention is thus highly advantageous because it is not excessively viscous owing to a DNAzyme concentration of under 75 mg/ml and can therefore be easily administered as an aerosol.

What is provided in particular is that the composition has a DNAzyme concentration of from 20 mg/ml to 50 mg/ml. Such a composition ensures that sufficient amounts of active ingredient can be taken up by the patient even when using manual inhalers or comparable inhalers. In the case of compositions having DNAzyme concentrations of under 20 mg/ml, there is namely the risk that the active-ingredient amounts released by an individual actuation or pressing of an inhaler are insufficient for bringing about the desired success of treatment—there are simply not enough active ingredients released per actuation of the inhaler. Consequently, patients have to press the inhaler multiple times, which, firstly, is a possible burden for the patients to be treated and, secondly, causes higher wear and tear of the inhalers. A high active-ingredient concentration also means that multiple administrations per inhaler are possible.

The composition according to the invention thus ensures especially an ideal compromise in this preferred embodiment: firstly, the DNAzyme concentration with less than 50 mg/ml is still low enough to still be able to be atomized by an inhaler and, secondly, the DNAzyme concentration with more than 20 mg/ml is high enough that, for example, sufficient active-ingredient amounts can be released after just a few presses when using manual inhalers.

According to an advantageous further development, what can also be provided is that the composition has a viscosity lower than 3.5 mPa·s. Such a viscosity means that the composition can be administered as an aerosol, for instance as an inhalation solution of an inhaler, in a problem-free manner and without significant loss of active ingredient. A higher viscosity means that the pressure which must be expended for atomization of the composition is too high to still be able to be exerted by a customary inhaler. Thus, a composition having a viscosity of over 3.5 mPa·s can lead to the atomizers of the inhalers usually used for treating corresponding patients blocking up or clogging up after just a short time. It is thus advantageous that the viscosity of the composition is within a range which ensures a highest possible active-ingredient concentration (DNAzymes) at a comparatively low viscosity.

What are mentioned as DNAzymes by way of example, but not definitively, and incorporated in this application are the DNAzymes of the earlier application DE 103 46 487. Said DNAzymes are directed against the mRNA of the proteins GATA-3 and T-bet and disclosed for production of an agent against inflammatory diseases.

For example, the following DNAzymes are used (each individually or in combination with the others):

Name of the DNAzymes against GATA-3 mRNA sequence:

hgd1
SEQ ID No: 1
5′-TCGGTCAGAggctagctacaacgaTGCGTTGCT-3′
hgd2
SEQ ID No: 2
5′-GGCGTACGAggctagctacaacgaCTGCTCGGT-3′
hgd3
SEQ ID No: 3
5′-GGCGGCGTAggctagctacaacgaGACCTGCTC-3′
hgd4
SEQ ID No: 4
5′-CTCGGGTCAggctagctacaacgaCTGGGTAGC-3′
hgd5
SEQ ID No: 5
5′-TCCTCTGCAggctagctacaacgaCGGGGTCCT-3′
hgd6
SEQ ID No: 6
5′-ACTCTGCAAggctagctacaacgaTCTGCGAGC-3′
hgd7
SEQ ID No: 7
5′-GGGCGACGAggctagctacaacgaTCTGCAATT-3′
hgd8
SEQ ID No: 8
5′-AAGGGGCGAggctagctacaacgaGACTCTGCA-3′
hgd9
SEQ ID No: 9
5′-AAAACGGGAggctagctacaacgaCAGGTTGTA-3′
hgd10
SEQ ID No: 10
5′-AGAATAAAAggctagctacaacgaGGGACCAGG-3′
hgd11
SEQ ID No: 11
5′-ATGGCAGAAggctagctacaacgaAAAACGGGA-3′
hgd12
SEQ ID No: 12
5′-AACTGGGTAggctagctacaacgaGGCAGAATA-3′
hgd13
SEQ ID No: 13
5′-ATCCAAAAAggctagctacaacgaTGGGTATGG-3′
hgd14
SEQ ID No: 14
5′-AGGGGAAGAggctagctacaacgaAAAAATCCA-3′
hgd15
SEQ ID No: 15
5′-TTTTAAAAAggctagctacaacgaTATCTTGGA-3′
hgd16
SEQ ID No: 16
5′-GTGGGGGGAggctagctacaacgaGGGAAGGCT-3′
hgd17
SEQ ID No: 17
5′-GTTGAATGAggctagctacaacgaTTGCTTTCG-3′
hgd18
SEQ ID No: 18
5′-GTCGTTGAAggctagctacaacgaGATTTGCTT-3′
hgd19
SEQ ID No: 19
5′-GGCCCGGAAggctagctacaacgaCCGCGCGCG-3′
hgd20
SEQ ID No: 20
5′-TCACCTCCAggctagctacaacgaGGCCTCGGC-3′
hgd21
SEQ ID No: 21
5′-CCGCCGTCAggctagctacaacgaCTCCATGGC-3′
hgd22
SEQ ID No: 22
5′-GGTGGCTCAggctagctacaacgaCCAGCGCGG-3′
hgd23
SEQ ID No: 23
5′-CGTTGAGCAggctagctacaacgaGGCGGGGTG-3′
hgd24
SEQ ID No: 24
5′-CCGCGTCCAggctagctacaacgaGTAGGAGTG-3′
hgd25
SEQ ID No: 25
5′-CAGCGGGTAggctagctacaacgaTGCGCCGCG-3′
hgd26
SEQ ID No: 26
5′-AAAAGCACAggctagctacaacgaCTCCTCCGG-3′
hgd27
SEQ ID No: 27
5′-AAAAGCACAggctagctacaacgaCCACCTCCT-3′
hgd28
SEQ ID No: 28
5′-TAAAAAGCAggctagctacaacgaATCCACCTC-3′
hgd29
SEQ ID No: 29
5′-GACCGTCGAggctagctacaacgaGTTAAAAAG-3′
hgd30
SEQ ID No: 30
5′-TTGCCTTGAggctagctacaacgaCGTCGATGT-3′
hgd31
SEQ ID No: 31
5′-AGGGCGGGAggctagctacaacgaGTGGTTGCC-3′
hgd32
SEQ ID No: 32
5′-TGGCCCTGAggctagctacaacgaCGAGTTTCC-3′
hgd33
SEQ ID No: 33
5′-ACCTCTGCAggctagctacaacgaCGTGGCCCT-3′
hgd34
SEQ ID No: 34
5′-CGGAGGGTAggctagctacaacgaCTCTGCACC-3′
hgd35
SEQ ID No: 35
5′-GGCGGCACAggctagctacaacgaCTGGCTCCC-3′
hgd36
SEQ ID No: 36
5′-CGGGCGGCAggctagctacaacgaACCTGGCTC-3′
hgd37
SEQ ID No: 37
5′-AGGGATCCAggctagctacaacgaGAAGCAGAG-3′
hgd38
SEQ ID No: 38
5′-GGGTAGGGAggctagctacaacgaCCATGAAGC-3′
hgd39
SEQ ID No: 39
5′-GGGCTGAGAggctagctacaacgaTCCAGGGGG-3′
hgd40
SEQ ID No: 40
5′-GTGGATGGAggctagctacaacgaGTCTTGGAG-3′
hgd41
SEQ ID No: 41
5′-CGTGGTGGAggctagctacaacgaGGACGTCTT-3′
hgd42
SEQ ID No: 42
5′-GGGGGTAGAggctagctacaacgaGGAGAGGGG-3′
hgd43
SEQ ID No: 43
5′-GGAGGAGGAggctagctacaacgGAGGCCGGGa-3′
hgd44
SEQ ID No: 44
5′-GCCCCCCGAggctagctacaacgaAAGGAGGAG-3′
hgd45
SEQ ID No: 45
5′-CCGGGGAGAggctagctacaacgaGTCCTTCGG-3′
hgd46
SEQ ID No: 46
5′-GGACAGCGAggctagctacaacgaGGGTCCGGG-3′
hgd47
SEQ ID No: 47
5′-TGGGGTGGAggctagctacaacgaAGCGATGGG-3′
hgd48
SEQ ID No: 48
5′-CTTGAGGCAggctagctacaacgaTCTTTCTCG-3′
hgd49
SEQ ID No: 49
5′-CACCTGGTAggctagctacaacgaTTGAGGCAC-3′
hgd50
SEQ ID No: 50
5′-GCAGGGGCAggctagctacaacgaCTGGTACTT-3′
hgd51
SEQ ID No: 51
5′-CCAGCTTCAggctagctacaacgaGCTGTCGGG-3′
hgd52
SEQ ID No: 52
5′-GTGGGACGAggctagctacaacgaTCCAGCTTC-3′
hgd53
SEQ ID No: 53
5′-GGAGTGGGAggctagctacaacgaGACTCCAGC-3′
hgd54
SEQ ID No: 54
5′-ATGCTGCCAggctagctacaacgaGGGAGTGGG-3′
hgd55
SEQ ID No: 55
5′-GGGCGGTCAggctagctacaacgaGCTGCCACG-3′
hgd56
SEQ ID No: 56
5′-GAGGCTCCAggctagctacaacgaCCAGGGCGG-3′
hgd57
SEQ ID No: 57
5′-GTGGGTCGAggctagctacaacgaGAGGAGGCT-3′
hgd58
SEQ ID No: 58
5′-AGGTGGTGAggctagctacaacgaGGGGTGGTG-3′
hgd59
SEQ ID No: 59
5′-ACTCGGGCAggctagctacaacgaGTAGGGCGG-3′
hgd60
SEQ ID No: 60
5′-GGAGCTGTAggctagctacaacgaTCGGGCACG-3′
hgd61
SEQ ID No: 61
5′-GGACTTGCAggctagctacaacgaCCGAAGCCG-3′
hgd62
SEQ ID No: 62
5′-GGGCCTGGAggctagctacaacgaTTGCATCCG-3′
hgd63
SEQ ID No: 63
5′-TGTGCTGGAggctagctacaacgaCGGGCCTTG-3′
hgd64
SEQ ID No: 64
5′-GTTCACACAggctagctacaacgaTCCCTGCCT-3′
hgd65
SEQ ID No: 65
5′-CAGTTCACAggctagctacaacgaACTCCCTGC-3′
hgd66
SEQ ID No: 66
5′-CACAGTTCAggctagctacaacgaACACTCCCT-3′
hgd67
SEQ ID No: 67
5′-GTTGCCCCAggctagctacaacgaAGTTCACAC-3′
hgd68
SEQ ID No: 68
5′-TCGCCGCCAggctagctacaacgaAGTGGGGTC-3′
hgd69
SEQ ID No: 69
5′-CCCGTGCCAggctagctacaacgaCTCGCCGCC-3′
hgd70
SEQ ID No: 70
5′-GGCGTTGCAggctagctacaacgaAGGTAGTGT-3′

Name of the DNAzymes against T-bet mRNA sequence:

td1
SEQ ID No: 71
5′-TGGCTTCTAggctagctacaacgaGCCCTCGTC-3′
td2
SEQ ID No: 72
5′-GGGCTCTGAggctagctacaacgaGCCTGGCTT-3′
td3
SEQ ID No: 73
5′-GGGACCCCAggctagctacaacgaCGGAGCCCG-3′
td4
SEQ ID No: 74
5′-GGTGGGGGAggctagctacaacgaCCCACCGGA-3′
td5
SEQ ID No: 75
5′-GGCGGGGGAggctagctacaacgaCCGAGGGCC-3′
td6
SEQ ID No: 76
5′-GGGCTGGGAggctagctacaacgaGGGCAGGGA-3′
td7
SEQ ID No: 77
5′-CGTCGAGGAggctagctacaacgaCCGCCCCTC-3′
td8
SEQ ID No: 78
5′-GGGCTGGCAggctagctacaacgaCTTCCCGTA-3′
td9
SEQ ID No: 79
5′-CGATGCCCAggctagctacaacgaCCGGGGCGG-3′
td10
SEQ ID No: 80
5′-GCTCCACGAggctagctacaacgaGCCCATCCG-3′
td11
SEQ ID No: 81
5′-CCGGCTCCAggctagctacaacgaGATGCCCAT-3′
td12
SEQ ID No: 82
5′-TCTCCGCAAggctagctacaacgaCCGGCTCCA-3′
td13
SEQ ID No: 83
5′-CCGTCAGCAggctagctacaacgaGTCTCCGCA-3′
td14
SEQ ID No: 84
5′-TCCCCGCCAggctagctacaacgaCGGCTCGGT-3′
td15
SEQ ID No: 85
5′-CCCCCGCGAggctagctacaacgaGCTCGTCCG-3′
td16
SEQ ID No: 86
5′-GTAGGGAGAggctagctacaacgaCCCAGGCTG-3′
td17
SEQ ID No: 87
5′-GGGCGGGCAggctagctacaacgaCAAGGCGCC-3′
td18
SEQ ID No: 88
5′-CGGGAAGGAggctagctacaacgaTCGCCCGCG-3′
td19
SEQ ID No: 89
5′-TAGTCCTCAggctagctacaacgaGCGGCCCCG-3′
td20
SEQ ID No: 90
5′-TCCCCGACAggctagctacaacgaCTCCAGTCC-3′
td21
SEQ ID No: 91
5′-TTTCCCCGAggctagctacaacgaACCTCCAGT-3′
td22
SEQ ID No: 92
5′-TGAGCGCGAggctagctacaacgaCCTCAGTTT-3′
td23
SEQ ID No: 93
5′-GGACCACAAggctagctacaacgaAGGTGGTTG-3′
td24
SEQ ID No: 94
5′-CTTGGACCAggctagctacaacgaAACAGGTGG-3′
td25
SEQ ID No: 95
5′-AAACTTGGAggctagctacaacgaCACAACAGG-3′
td26
SEQ ID No: 96
5′-CTGATTAAAggctagctacaacgaTTGGACCAC-3′
td27
SEQ ID No: 97
5′-TGGTGCTGAggctagctacaacgaTAAACTTGG-3′
td28
SEQ ID No: 98
5′-TGATGATCAggctagctacaacgaCTCTGTCTG-3′
td29
SEQ ID No: 99
5′-TGGTGATGAggctagctacaacgaCATCTCTGT-3′
td30
SEQ ID No: 100
5′-GCTTGGTGAggctagctacaacgaGATCATCTC-3′
td31
SEQ ID No: 101
5′-ATGGGAACAggctagctacaacgaCCGCCGTCC-3′
td32
SEQ ID No: 102
5′-GAATGGGAAggctagctacaacgaATCCGCCGT-3′
td33
SEQ ID No: 103
5′-TGACAGGAAggctagctacaacgaGGGAACATC-3′
td34
SEQ ID No: 104
5′-AGTAAATGAggctagctacaacgaAGGAATGGG-3′
td35
SEQ ID No: 105
5′-CACAGTAAAggctagctacaacgaGACAGGAAT-3′
td36
SEQ ID No: 106
5′-GCCCGGCCAggctagctacaacgaAGTAAATGA-3′
td37
SEQ ID No: 107
5′-CCACAAACAggctagctacaacgaCCTGTAGTG-3′
td38
SEQ ID No: 108
5′-GTCCACAAAggctagctacaacgaATCCTGTAG-3′
td39
SEQ ID No: 109
5′-CCACGTCCAggctagctacaacgaAAACATCCT-3′
td40
SEQ ID No: 110
5′-CCAAGACCAggctagctacaacgaGTCCACAAA-3′
td41
SEQ ID No: 111
5′-CCACCAAGAggctagctacaacgaCACGTCCAC-3′
td42
SEQ ID No: 112
5′-GCTGGTCCAggctagctacaacgaCAAGACCAC-3′
td43
SEQ ID No: 113
5′-GCTCTGGTAggctagctacaacgaCGCCAGTGG-3′
td44
SEQ ID No: 114
5′-CTGCACCCAggctagctacaacgaTTGCCGCTC-3′
td45
SEQ ID No: 115
5′-CACACTGCAggctagctacaacgaCCACTTGCC-3′
td46
SEQ ID No: 116
5′-CTTTCCACAggctagctacaacgaTGCACCCAC-3′
td47
SEQ ID No: 117
5′-GCCTTTCCAggctagctacaacgaACTGCACCC-3′
td48
SEQ ID No: 118
5′-TTCCTGGCAggctagctacaacgaGCTGCCCTC-3′
td49
SEQ ID No: 119
5′-GTGGACGTAggctagctacaacgaAGGCGGTTT-3′
td50
SEQ ID No: 120
5′-CCGGGTGGAggctagctacaacgaGTACAGGCG-3′
td51
SEQ ID No: 121
5′-CCTGGCGCAggctagctacaacgaCCAGTGCGC-3′
td52
SEQ ID No: 122
5′-CAAATGAAAggctagctacaacgaTTCCTGGCG-3′
td53
SEQ ID No: 123
5′-TTTCCCAAAggctagctacaacgaGAAACTTCC-3′
td54
SEQ ID No: 124
5′-ATTGTTGGAggctagctacaacgaGCCCCCTTG-3′
td55
SEQ ID No: 125
5′-TGGGTCACAggctagctacaacgaTGTTGGACG-3′
td56
SEQ ID No: 126
5′-TCTGGGTCAggctagctacaacgaATTGTTGGA-3′
td57
SEQ ID No: 127
5′-GCACAATCAggctagctacaacgaCTGGGTCAC-3′
td58
SEQ ID No: 128
5′-GGAGCACAAggctagctacaacgaCATCTGGGT-3′
td59
SEQ ID No: 129
5′-ACTGGAGCAggctagctacaacgaAATCATCTG-3′
td60
SEQ ID No: 130
5′-ATGGAGGGAggctagctacaacgaTGGAGCACA-3′
td61
SEQ ID No: 131
5′-TGGTACTTAggctagctacaacgaGGAGGGACT-3′
td62
SEQ ID No: 132
5′-GGGCTGGTAggctagctacaacgaTTATGGAGG-3′
td63
SEQ ID No: 133
5′-TCAACGATAggctagctacaacgaGCAGCCGGG-3′
td64
SEQ ID No: 134
5′-CCTCAACGAggctagctacaacgaATGCAGCCG-3′
td65
SEQ ID No: 135
5′-TCACCTCAAggctagctacaacgaGATATGCAG-3′
td66
SEQ ID No: 136
5′-CGTCGTTCAggctagctacaacgaCTCAACGAT-3′
td67
SEQ ID No: 137
5′-GTAAAGATAggctagctacaacgaGCGTGTTGG-3′
td68
SEQ ID No: 138
5′-AAGTAAAGAggctagctacaacgaATGCGTGTT-3′
td69
SEQ ID No: 139
5′-GGCAATGAAggctagctacaacgaTGGGTTTCT-3′
td70
SEQ ID No: 140
5′-TCACGGCAAggctagctacaacgaGAACTGGGT-3′
td71
SEQ ID No: 141
5′-AGGCAGTCAggctagctacaacgaGGCAATGAA-3′
td72
SEQ ID No: 142
5′-ATCTCGGCAggctagctacaacgaTCTGGTAGG-3′
td73
SEQ ID No: 143
5′-GCTGAGTAAggctagctacaacgaCTCGGCATT-3′
td74
SEQ ID No: 144
5′-TATTATCAAggctagctacaacgaTTTCAGCTG-3′
td75
SEQ ID No: 145
5′-GGGTTATTAggctagctacaacgaCAATTTTCA-3′
td76
SEQ ID No: 146
5′-AAGGGGTTAggctagctacaacgaTATCAATTT-3′
td77
SEQ ID No: 147
5′-CTCCCGGAAggctagctacaacgaCCTTTGGCA-3′
td78
SEQ ID No: 148
5′-GTACATGGAggctagctacaacgaTCAAAGTCC-3′

Accordingly, what is provided by an advantageous embodiment is that the DNAzyme is selected from a group comprising the DNAzymes hgd1 to hgd70 (cf. also Table 2) and/or from a group comprising the DNAzymes td1 to td78 (cf. Table 2).

DNAzymes having one of these sequences prove to be particularly efficient for specific in vivo inhibition of the expressions of the inflammation-promoting transcription factors GATA-3 and Tbet. In general, preference is given in this connection to the sequences, the substrate-binding domains of which are completely complementary to the flanking regions of the cleavage site of a particular target mRNA. However, a DNAzyme does not have to be completely complementary in order to bind and cleave a target mRNA—what may be sufficient are smaller complementary sequence segments within the altogether homologous substrate-binding domain.

What is provided as a particularly preferred variant is a composition which comprises the DNAzyme hgd40 GTGGATGGAggctagctacaacgaGTCTTGGAG (SEQ ID No. 40) for downregulation of the expression of the transcription factor GATA-3.

This sequence exhibits particularly high enzyme activity and cleaves GATA-3 mRNA with high specificity and with high efficiency. Consequently, a DNAzyme having the sequence hgd40 (SEQ ID No. 40) is outstandingly suitable for specific and effective treatment of respiratory tract diseases which are Th2-dependent, i.e. which correspond to an increased GATA-3 level.

According to an advantageous further development, the composition can comprise at least one nuclease inhibitor, the at least one nuclease inhibitor specifically inactivating especially deoxyribonucleases, i.e. being a DNase inhibitor. By means of such a nuclease inhibitor, the at least one DNAzyme is protected from enzymatic degradation which can be caused by DNases.

It is also advantageous when the composition comprises at least one salt and/or at least one cation.

Such a composition is advantageously suitable for use in the therapy of patients suffering from a respiratory tract disease associated with chronic inflammations because the composition has a physiologically favorable environment as a result and is thus readily compatible for patients. In this connection, what is provided in particular is a phosphate-buffered saline solution (PBS). However, it is also possible to provide other buffered solutions in which nucleic acids can be present in a dissolved state in a physiologically favorable environment, such as TE buffer for example. Accordingly, the inorganic and/or organic additive is preferably selected from the group comprising the substances sodium chloride (NaCl), potassium chloride (KCl), disodium hydrogenphosphate (Na₂HPO₄), disodium hydrogenphosphate dihydrate, potassium dihydrogenphosphate (KH₂PO₄), TRIS and EDTA. Said cation can be selected from the group comprising Na, Mg, K, Li, Ca, Fe, Cu and Ag. Furthermore, it is also possible to provide an organic cation, such as, for example, Mg(N(SO₂CF3)₂)₂ or Mg(OSO₂CF₃)₂. Furthermore, it is possible to provide a bivalent cation.

Such a buffered composition is particularly suitable for protecting the DNAzymes because the DNAzyme is stabilized by the inorganic and/or organic additive and protected from enzymatic degradation. Furthermore, the salt allows a good uptake into the target cells. The bivalent cations can, as cofactors of the DNAzymes, increase DNAzyme activity, since the DNAzymes have a catalytic domain dependent on bivalent cations (preferably Mg2+). Thus, bivalent cations act as enhancers or boosters.

According to an advantageous further development, the composition can comprise at least one inorganic and/or organic additive and/or a solubilizer and/or a preservative. Said solubilizer serves especially for complex formation. Furthermore, it improves the solvent properties of the composition. What can be provided as solubilizers are preferably glycerol derivatives and/or polyethylene glycols or else lecithins. The preservative can, for example, be paraben. Furthermore, it is possible to provide additives which, for example, bring about a change in the surface tension of the composition or a lowering of the viscosity of the composition.

An additional aspect of this invention is the method for producing the composition according to the invention, it being provided that the method comprises at least the following steps:
a) preparing a DNAzyme-containing solution, the DNAzyme concentration in the aqueous solution not exceeding 80 mg/ml;
b) filtering the solution from step a) until the solution has a DNAzyme concentration of under 75 mg/ml.

The filtration step is advantageous because undesired (nonsterile) substances and undissolved active-ingredient particles (i.e. undissolved DNAzymes) are removed as a result. The composition must be sterile for treatment of a patient suffering from a respiratory tract disease associated with chronic inflammations. Other sterilization methods (radiation, heating, autoclaving, etc.) are not usable here because the DNAzymes would also be destroyed thereby. For this reason, what is provided is that the solution is sterilized by means of filtration. This preferably involves using a filter which comprises a PES membrane having a pore diameter of from 0.20 to 0.25 μm. Particular preference is given to a PES membrane having a pore diameter of 0.22 μm.

According to a further embodiment, what is provided is that the method comprises at least one further method step, namely selected from one of the following steps:
adding a nuclease inhibitor to the solution from step b);
adding the at least one salt and/or the at least one cation to the solution from step b);
adding the inorganic and/or organic additive to the solution from step b);
adding the solubilizer and/or the preservative to the solution from step b).

Such a method ensures, in a simple manner, the production of a composition which can be used for treating a patient suffering from a respiratory tract disease associated with chronic inflammations. By selecting the steps, it is possible to individually adapt the method to specific requirements.

A further aspect of the invention is provided by the use of the aforementioned composition as a drug for treating a patient suffering from a respiratory tract disease associated with chronic inflammations. To this end, the composition can be present especially as an inhalation solution. However, other forms are also conceivable, such as, for instance, a powder for use in powder inhalers.

In a preferred embodiment, what is provided is that the composition can be administered in the form of an aerosol. Such an administered composition can act in a site-specific manner in the lungs of a patient suffering from a respiratory tract disease associated with chronic inflammations. This can increase the effectiveness of the drug. At the same time, the risk of adverse effects is minimized.

Further features, details and advantages of the invention become apparent from the wording of the claims and from the following description of exemplary embodiments, where:

Table 1 shows a list of the GATA-3-specific DNAzymes;

Table 2 shows a list of the Tbet-specific DNAzymes;

Table 3 shows an example of a composition according to the invention;

Table 4 shows possible additional components of the composition from Table 3;

FIG. 1 shows a graphic representation of the viscosity of a DNAzyme-containing composition according to its concentration;

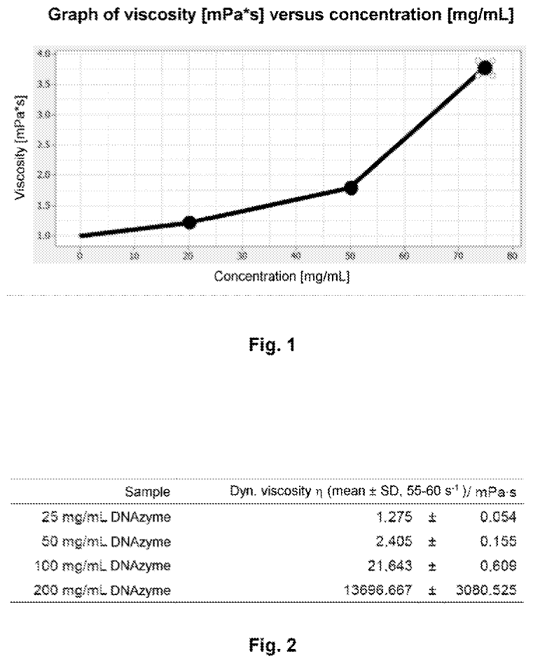

FIG. 2 shows viscosity measurement results for a DNAzyme-containing composition according to its concentration;

FIG. 3 shows measurement results for a FAT of compositions having different DNAzyme concentrations;

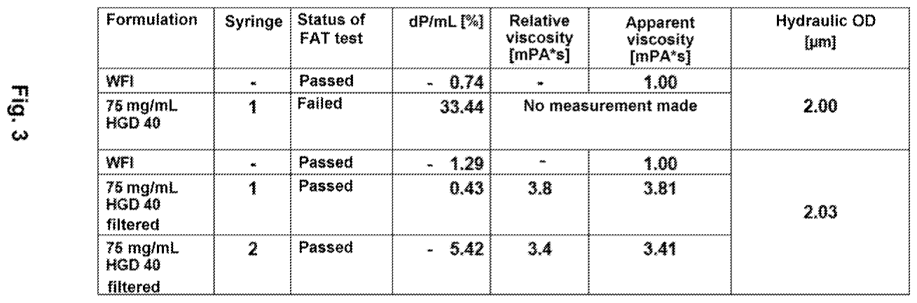

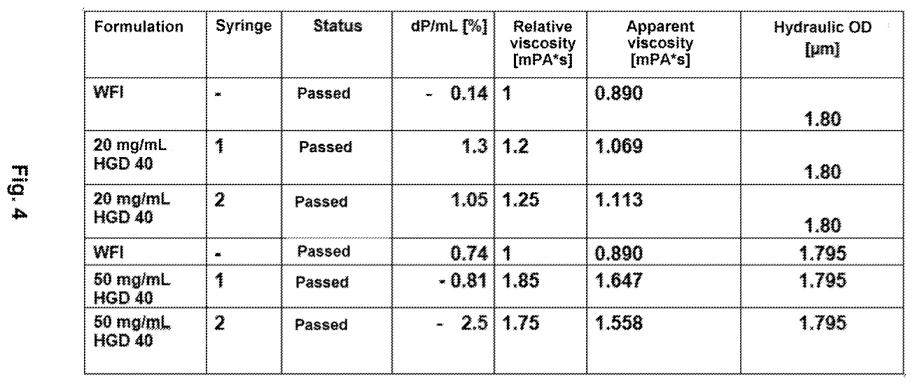

FIG. 4 shows measurement results for a FAT of the composition according to the invention having DNAzyme concentrations of 20 mg/ml or 50 mg/ml.

### EXEMPLARY EMBODIMENTS

TABLE 1
GATA-3-specific DNAzymes

| SEQ ID | Name | Sequence |
|---|---|---|
| SEQ ID No: 1 | hgd1 | 5′-TCGGTCAGAggctagctacaacgaTGC |
| | | GTTGCT-3′ |
| SEQ ID No: 2 | hgd2 | 5′-GGCGTACGAggctagctacaacgaCTG |
| | | CTCGGT-3′ |
| SEQ ID No: 3 | hgd3 | 5′-GGCGGCGTAggctagctacaacgaGAC |
| | | CTGCTC-3′ |
| SEQ ID No: 4 | hgd4 | 5′-CTCGGGTCAggctagctacaacgaCTG |
| | | GGTAGC-3′ |
| SEQ ID No: 5 | hgd5 | 5′-TCCTCTGCAggctagctacaacgaCGG |
| | | GGTCCT-3′ |
| SEQ ID No: 6 | hgd6 | 5′-ACTCTGCAAggctagctacaacgaTCT |
| | | GCGAGC-3′ |
| SEQ ID No: 7 | hgd7 | 5′-GGGCGACGAggctagctacaacgaTCT |
| | | GCAATT-3′ |
| SEQ ID No: 8 | hgd8 | 5′-AAGGGGCGAggctagctacaacgaGAC |
| | | TCTGCA-3′ |
| SEQ ID No: 9 | hgd9 | 5′-AAAACGGGAggctagctacaacgaCAG |
| | | GTTGTA-3′ |
| SEQ ID No: 10 | hgd10 | 5′-AGAATAAAAggctagctacaacgaGGG |
| | | ACCAGG-3′ |
| SEQ ID No: 11 | hgd11 | 5′-ATGGCAGAAggctagctacaacgaAAA |
| | | ACGGGA-3′ |
| SEQ ID No: 12 | hgd12 | 5′-AACTGGGTAggctagctacaacgaGGC |
| | | AGAATA-3′ |
| SEQ ID No: 13 | hgd13 | 5′-ATCCAAAAAggctagctacaacgaTGG |
| | | GTATGG-3′ |
| SEQ ID No: 14 | hgd14 | 5′-AGGGGAAGAggctagctacaacgaAAA |
| | | AATCCA-3′ |
| SEQ ID No: 15 | hgd15 | 5′-TTTTAAAAAggctagctacaacgaTAT |
| | | CTTGGA-3′ |
| SEQ ID No: 16 | hgd16 | 5′-GTGGGGGGAggctagctacaacgaGGG |
| | | AAGGCT-3′ |
| SEQ ID No: 17 | hgd17 | 5′-GTTGAATGAggctagctacaacgaTTG |
| | | CTTTCG-3′ |
| SEQ ID No: 18 | hgd18 | 5′-GTCGTTGAAggctagctacaacgaGAT |
| | | TTGCTT-3′ |
| SEQ ID No: 19 | hgd19 | 5′-GGCCCGGAAggctagctacaacgaCCG |
| | | CGCGCG-3′ |
| SEQ ID No: 20 | hgd20 | 5′-TCACCTCCAggctagctacaacgaGGC |
| | | CTCGGC-3′ |
| SEQ ID No: 21 | hgd21 | 5′-CCGCCGTCAggctagctacaacgaCTC |
| | | CATGGC-3′ |
| SEQ ID No: 22 | hgd22 | 5′-GGTGGCTCAggctagctacaacgaCCA |
| | | GCGCGG-3′ |
| SEQ ID No: 23 | hgd23 | 5′-CGTTGAGCAggctagctacaacgaGGC |
| | | GGGGTG-3′ |
| SEQ ID No: 24 | hgd24 | 5′-CCGCGTCCAggctagctacaacgaGTA |
| | | GGAGTG-3′ |
| SEQ ID No: 25 | hgd25 | 5′-CAGCGGGTAggctagctacaacgaTGC |
| | | GCCGCG-3′ |
| SEQ ID No: 26 | hgd26 | 5′-GCACATCCAggctagctacaacgaCTC |
| | | CTCCGG-3′ |
| SEQ ID No: 27 | hgd27 | 5′-AAAAGCACAggctagctacaacgaCCA |
| | | CCTCCT-3′ |
| SEQ ID No: 28 | hgd28 | 5′-TAAAAAGCAggctagctacaacgaATC |
| | | CACCTC-3′ |
| SEQ ID No: 29 | hgd29 | 5′-GACCGTCGAggctagctacaacgaGTT |
| | | AAAAAG-3′ |
| SEQ ID No: 30 | hgd30 | 5′-TTGCCTTGAggctagctacaacgaCGT |
| | | CGATGT-3′ |
| SEQ ID No: 31 | hgd31 | 5′-AGGGCGGGAggctagctacaacgaGTG |
| | | GTTGCC-3′ |
| SEQ ID No: 32 | hgd32 | 5′-TGGCCCTGAggctagctacaacgaCGA |
| | | GTTTCC-3′ |
| SEQ ID No: 33 | hgd33 | 5′-ACCTCTGCAggctagctacaacgaCGT |
| | | GGCCCT-3′ |
| SEQ ID No: 34 | hgd34 | 5′-CGGAGGGTAggctagctacaacgaCTC |
| | | TGCACC-3′ |
| SEQ ID No: 35 | hgd35 | 5′-GGCGGCACAggctagctacaacgaCTG |
| | | GCTCCC-3′ |
| SEQ ID No: 36 | hgd36 | 5′-CGGGCGGCAggctagctacaacgaACC |
| | | TGGCTC-3′ |
| SEQ ID No: 37 | hgd37 | 5′-AGGGATCCAggctagctacaacgaGAA |
| | | GCAGAG-3′ |
| SEQ ID No: 38 | hgd38 | 5′-GGGTAGGGAggctagctacaacgaCCA |
| | | TGAAGC-3′ |
| SEQ ID No: 39 | hgd39 | 5′-GGGCTGAGAggctagctacaacgaTCC |
| | | AGGGGG-3′ |
| SEQ ID No: 40 | hgd40 | 5′-GTGGATGGAggctagctacaacgaGTC |
| | | TTGGAG-3′ |
| SEQ ID No: 41 | hgd41 | 5′-CGTGGTGGAggctagctacaacgaGGA |
| | | CGTCTT-3′ |
| SEQ ID No: 42 | hgd42 | 5′-GGGGGTAGAggctagctacaacgaGGA |
| | | GAGGGG-3′ |
| SEQ ID No: 43 | hgd43 | 5′-GGAGGAGGAggctagctacaacgaGAG |
| | | GCCGGG-3′ |
| SEQ ID No: 44 | hgd44 | 5′-GCCCCCCGAggctagctacaacgaAAG |
| | | GAGGAG-3′ |
| SEQ ID No: 45 | hgd45 | 5′-CCGGGGAGAggctagctacaacgaGTC |
| | | CTTCGG-3′ |
| SEQ ID No: 46 | hgd46 | 5′-GGACAGCGAggctagctacaacgaGGG |
| | | TCCGGG-3′ |
| SEQ ID No: 47 | hgd47 | 5′-TGGGGTGGAggctagctacaacgaAGC |
| | | GATGGG-3′ |
| SEQ ID No: 48 | hgd48 | 5′-CTTGAGGCAggctagctacaacgaTCT |
| | | TTCTCG-3′ |
| SEQ ID No: 49 | hgd49 | 5′-CACCTGGTAggctagctacaacgaTTG |
| | | AGGCAC-3′ |
| SEQ ID No: 50 | hgd50 | 5′-GCAGGGGCAggctagctacaacgaCTG |
| | | GTACTT-3′ |
| SEQ ID No: 51 | hgd51 | 5′-CCAGCTTCAggctagctacaacgaGCT |
| | | GTCGGG-3′ |
| SEQ ID No: 52 | hgd52 | 5′-GTGGGACGAggctagctacaacgaTCC |
| | | AGCTTC-3′ |
| SEQ ID No: 53 | hgd53 | 5′-GGAGTGGGAggctagctacaacgaGAC |
| | | TCCAGC-3′ |
| SEQ ID No: 54 | hgd54 | 5′-ATGCTGCCAggctagctacaacgaGGG |
| | | AGTGGG-3′ |
| SEQ ID No: 55 | hgd55 | 5′-GGGCGGTCAggctagctacaacgaGCT |
| | | GCCACG-3′ |
| SEQ ID No: 56 | hgd56 | 5′-GAGGCTCCAggctagctacaacgaCCA |
| | | GGGCGG-3′ |
| SEQ ID No: 57 | hgd57 | 5′-GTGGGTCGAggctagctacaacgaGAG |
| | | GAGGCT-3′ |
| SEQ ID No: 58 | hgd58 | 5′-AGGTGGTGAggctagctacaacgaGGG |
| | | GTGGTG-3′ |
| SEQ ID No: 59 | hgd59 | 5′-ACTCGGGCAggctagctacaacgaGTA |
| | | GGGCGG-3′ |
| SEQ ID No: 60 | hgd60 | 5′-GGAGCTGTAggctagctacaacgaTCG |
| | | GGCACG-3′ |
| SEQ ID No: 61 | hgd61 | 5′-GGACTTGCAggctagctacaacgaCCG |
| | | AAGCCG-3′ |
| SEQ ID No: 62 | hgd62 | 5′-GGGCCTGGAggctagctacaacgaTTG |
| | | CATCCG-3′ |
| SEQ ID No: 63 | hgd63 | 5′-TGTGCTGGAggctagctacaacgaCGG |
| | | GCCTTG-3′ |
| SEQ ID No: 64 | hgd64 | 5′-GTTCACACAggctagctacaacgaTCC |
| | | CTGCCT-3′ |
| SEQ ID No: 65 | hgd65 | 5′-CAGTTCACAggctagctacaacgaACT |
| | | CCCTGC-3′ |
| SEQ ID No: 66 | hgd66 | 5′-CACAGTTCAggctagctacaacgaACA |
| | | CTCCCT-3′ |
| SEQ ID No: 67 | hgd67 | 5′-GTTGCCCCAggctagctacaacgaAGT |
| | | TCACAC-3′ |
| SEQ ID No: 68 | hgd68 | 5′-TCGCCGCCAggctagctacaacgaAGT |
| | | GGGGTC-3′ |
| SEQ ID No: 69 | hgd69 | 5′-CCCGTGCCAggctagctacaacgaCTC |
| | | GCCGCC-3′ |
| SEQ ID No: 70 | hgd70 | 5′-GGCGTTGCAggctagctacaacgaAGG |
| | | TAGTGT-3′ |

TABLE 2
Tbet-specific DNAzymes

| SEQ ID | Name | Sequence |
|---|---|---|
| SEQ ID No: 71 | td1 | 5′-TGGCTTCTAggctagctacaacgaGCC |
| | | CTCGTC-3′ |
| SEQ ID No: 72 | td2 | 5′-GGGCTCTGAggctagctacaacgaGCC |
| | | TGGCTT-3′ |
| SEQ ID No: 73 | td3 | 5′-GGGACCCCAggctagctacaacgaCGG |
| | | AGCCCG-3′ |
| SEQ ID No: 74 | td4 | 5′-GGTGGGGGAggctagctacaacgaCCC |
| | | ACCGGA-3′ |
| SEQ ID No: 75 | td5 | 5′-GGCGGGGGAggctagctacaacgaCCG |
| | | AGGGCC-3′ |
| SEQ ID No: 76 | td6 | 5′-GGGCTGGGAggctagctacaacgaGGG |
| | | CAGGGA-3′ |
| SEQ ID No: 77 | td7 | 5′-CGTCGAGGAggctagctacaacgaCCG |
| | | CCCCTC-3′ |
| SEQ ID No: 78 | td8 | 5′-GGGCTGGCAggctagctacaacgaCTT |
| | | CCCGTA-3′ |
| SEQ ID No: 79 | td9 | 5′-CGATGCCCAggctagctacaacgaCCG |
| | | GGGCGG-3′ |
| SEQ ID No: 80 | td10 | 5′-GCTCCACGAggctagctacaacgaGCC |
| | | CATCCG-3′ |
| SEQ ID No: 81 | td11 | 5′-CCGGCTCCAggctagctacaacgaGAT |
| | | GCCCAT-3′ |
| SEQ ID No: 82 | td12 | 5′-TCTCCGCAAggctagctacaacgaCCG |
| | | GCTCCA-3′ |
| SEQ ID No: 83 | td13 | 5′-CCGTCAGCAggctagctacaacgaGTC |
| | | TCCGCA-3′ |
| SEQ ID No: 84 | td14 | 5′-TCCCCGGCAggctagctacaacgaCGG |
| | | CTCGGT-3′ |
| SEQ ID No: 85 | td15 | 5′-CCCCCGCGAggctagctacaacgaGCT |
| | | CGTCCG-3′ |
| SEQ ID No: 86 | td16 | 5′-GTAGGGAGAggctagctacaacgaCCC |
| | | AGGCTG-3′ |
| SEQ ID No: 87 | td17 | 5′-GGGCGGGCAggctagctacaacgaCAA |
| | | GGCGCC-3′ |
| SEQ ID No: 88 | td18 | 5′-CGGGAAGGAggctagctacaacgaTCG |
| | | CCCGCG-3′ |
| SEQ ID No: 89 | td19 | 5′-TAGTCCTCAggctagctacaacgaGCG |
| | | GCCCCG-3′ |
| SEQ ID No: 90 | td20 | 5′-TCCCCGACAggctagctacaacgaCTC |
| | | CAGTCC-3′ |
| SEQ ID No: 91 | td21 | 5′-TTTCCCCGAggctagctacaacgaACC |
| | | TCCAGT-3′ |
| SEQ ID No: 92 | td22 | 5′-TGAGCGCGAggctagctacaacgaCCT |
| | | CAGTTT-3′ |
| SEQ ID No: 93 | td23 | 5′-GGACCACAAggctagctacaacgaAGG |
| | | TGGTTG-3′ |
| SEQ ID No: 94 | td24 | 5′-CTTGGACCAggctagctacaacgaAAC |
| | | AGGTGG-3′ |
| SEQ ID No: 95 | td25 | 5′-AAACTTGGAggctagctacaacgaCAC |
| | | AACAGG-3′ |
| SEQ ID No: 96 | td26 | 5′-CTGATTAAAggctagctacaacgaTTG |
| | | GACCAC-3′ |
| SEQ ID No: 97 | td27 | 5′-TGGTGCTGAggctagctacaacgaTAA |
| | | ACTTGG-3′ |
| SEQ ID No: 98 | td28 | 5′-TGATGATCAggctagctacaacgaCTC |
| | | TGTCTG-3′ |
| SEQ ID No: 99 | td29 | 5′-TGGTGATGAggctagctacaacgaCAT |
| | | CTCTGT-3′ |
| SEQ ID No: 100 | td30 | 5′-GCTTGGTGAggctagctacaacgaGAT |
| | | CATCTC-3′ |
| SEQ ID No: 101 | td31 | 5′-ATGGGAACAggctagctacaacgaCCG |
| | | CCGTCC-3′ |
| SEQ ID No: 102 | td32 | 5′-GAATGGGAAggctagctacaacgaATC |
| | | CGCCGT-3′ |
| SEQ ID No: 103 | td33 | 5′-TGACAGGAAggctagctacaacgaGGG |
| | | AACATC-3′ |
| SEQ ID No: 104 | td34 | 5′-AGTAAATGAggctagctacaacgaAGG |
| | | AATGGG-3′ |
| SEQ ID No: 105 | td35 | 5′-CACAGTAAAggctagctacaacgaGAC |
| | | AGGAAT-3′ |
| SEQ ID No: 106 | td36 | 5′-GCCCGGCCAggctagctacaacgaAGT |
| | | AAATGA-3′ |
| SEQ ID No: 107 | td37 | 5′-CCACAAACAggctagctacaacgaCCT |
| | | GTAGTG-3′ |
| SEQ ID No: 108 | td38 | 5′-GTCCACAAAggctagctacaacgaATC |
| | | CTGTAG-3′ |
| SEQ ID No: 109 | td39 | 5′-CCACGTCCAggctagctacaacgaAAA |
| | | CATCCT-3′ |
| SEQ ID No: 110 | td40 | 5′-CCAAGACCAggctagctacaacgaGTC |
| | | CACAAA-3′ |
| SEQ ID No: 111 | td41 | 5′-CCACCAAGAggctagctacaacgaCAC |
| | | GTCCAC-3′ |
| SEQ ID No: 112 | td42 | 5′-GCTGGTCCAggctagctacaacgaCAA |
| | | GACCAC-3′ |
| SEQ ID No: 113 | td43 | 5′-GCTCTGGTAggctagctacaacgaCGC |
| | | CAGTGG-3′ |
| SEQ ID No: 114 | td44 | 5′-CTGCACCCAggctagctacaacgaTTG |
| | | CCGCTC-3′ |
| SEQ ID No: 115 | td45 | 5′-CACACTGCAggctagctacaacgaCCA |
| | | CTTGCC-3′ |
| SEQ ID No: 116 | td46 | 5′-CTTTCCACAggctagctacaacgaTGC |
| | | ACCCAC-3′ |
| SEQ ID No: 117 | td47 | 5′-GCCTTTCCAggctagctacaacgaACT |
| | | GCACCC-3′ |
| SEQ ID No: 118 | td48 | 5′-TTCCTGGCAggctagctacaacgaGCT |
| | | GCCCTC-3′ |
| SEQ ID No: 119 | td49 | 5′-GTGGACGTAggctagctacaacgaAGG |
| | | CGGTTT-3′ |
| SEQ ID No: 120 | td50 | 5′-CCGGGTGGAggctagctacaacgaGTA |
| | | CAGGCG-3′ |
| SEQ ID No: 121 | td51 | 5′-CCTGGCGCAggctagctacaacgaCCA |
| | | GTGCGC-3′ |
| SEQ ID No: 122 | td52 | 5′-CAAATGAAAggctagctacaacgaTTC |
| | | CTGGCG-3′ |
| SEQ ID No: 123 | td53 | 5′-TTTCCCAAAggctagctacaacgaGAA |
| | | ACTTCC-3′ |
| SEQ ID No: 124 | td54 | 5′-ATTGTTGGAggctagctacaacgaGCC |
| | | CCCTTG-3′ |
| SEQ ID No: 125 | td55 | 5′-TGGGTCACAggctagctacaacgaTGT |
| | | TGGACG-3′ |
| SEQ ID No: 126 | td56 | 5′-TCTGGGTCAggctagctacaacgaATT |
| | | GTTGGA-3′ |
| SEQ ID No: 127 | td57 | 5′-GCACAATCAggctagctacaacgaCTG |
| | | GGTCAC-3′ |
| SEQ ID No: 128 | td58 | 5′-GGAGCACAAggctagctacaacgaCAT |
| | | CTGGGT-3′ |
| SEQ ID No: 129 | td59 | 5′-ACTGGAGCAggctagctacaacgaAAT |
| | | CATCTG-3′ |
| SEQ ID No: 130 | td60 | 5′-ATGGAGGGAggctagctacaacgaTGG |
| | | AGCACA-3′ |
| SEQ ID No: 131 | td61 | 5′-TGGTACTTAggctagctacaacgaGGA |
| | | GGGACT-3′ |
| SEQ ID No: 132 | td62 | 5′-GGGCTGGTAggctagctacaacgaTTA |
| | | TGGAGG-3′ |
| SEQ ID No: 133 | td63 | 5′-TCAACGATAggctagctacaacgaGCA |
| | | GCCGGG-3′ |
| SEQ ID No: 134 | td64 | 5′-CCTCAACGAggctagctacaacgaATG |
| | | CAGCCG-3′ |
| SEQ ID No: 135 | td65 | 5′-TCACCTCAAggctagctacaacgaGAT |
| | | ATGCAG-3′ |
| SEQ ID No: 136 | td66 | 5′-CGTCGTTCAggctagctacaacgaCTC |
| | | AACGAT-3′ |
| SEQ ID No: 137 | td67 | 5′-GTAAAGATAggctagctacaacgaGCG |
| | | TGTTGG-3′ |
| SEQ ID No: 138 | td68 | 5′-AAGTAAAGAggctagctacaacgaATG |
| | | CGTGTT-3′ |
| SEQ ID No: 139 | td69 | 5′-GGCAATGAAggctagctacaacgaTGG |
| | | GTTTCT-3′ |
| SEQ ID No: 140 | td70 | 5′-TCACGGCAAggctagctacaacgaGAA |
| | | CTGGGT-3′ |
| SEQ ID No: 141 | td71 | 5′-AGGCAGTCAggctagctacaacgaGGC |
| | | AATGAA-3′ |
| SEQ ID No: 142 | td72 | 5′-ATCTCGGCAggctagctacaacgaTCT |
| | | GGTAGG-3′ |
| SEQ ID No: 143 | td73 | 5′-GCTGAGTAAggctagctacaacgaCTC |
| | | GGCATT-3′ |
| SEQ ID No: 144 | td74 | 5′-TATTATCAAggctagctacaacgaTTT |
| | | CAGCTG-3′ |
| SEQ ID No: 145 | td75 | 5′-GGGTTATTAggctagctacaacgaCAA |
| | | TTTTCA-3′ |
| SEQ ID No: 146 | td76 | 5′-AAGGGGTTAggctagctacaacgaTAT |
| | | CAATTT-3′ |
| SEQ ID No: 147 | td77 | 5′-CTCCCGGAAggctagctacaacgaCCT |
| | | TTGGCA-3′ |
| SEQ ID No: 148 | td78 | 5′-GTACATGGAggctagctacaacgaTCA |
| | | AAGTTC-3′ |

TABLE 3
Example of a composition according to the invention

| Substance | [% w/w] |
|---|---|
| DNAzyme | 0.01-0.75 |
| Nuclease inhibitor | variable |
| Example: DNase inhibitor | |

TABLE 4
Possible additional components of the composition from Table 3

| Substance | [% w/w] |
|---|---|
| Salt and/or cation | variable |
| Example: Na, Mg, K, Li, Ca, Fe, Cu, Ag, HPO₄²⁻, H₂PO₄⁻, | |
| EDTA | variable |
| TRIS | variable |
| Solubilizer | variable |
| Example: glycerol derivatives, polyethylene glycols, lecithins | |
| Preservative | variable |
| Example: paraben | |

FIG. 1 shows a graphic representation of the viscosity of a DNAzyme-containing composition according to its concentration. Said DNAzyme can specifically downregulate the expression of GATA-3 and/or specifically downregulate the expression of Tbet. According to a preferred embodiment, what are provided are especially DNAzymes from a group comprising the DNAzymes hgd1 to hgd70 or from a group comprising the DNAzymes td1 to td78. In particular, what is to be used is the DNAzyme hgd40 (SEQ ID No. 40), which has the sequence GTGGATGGAggctagctacaacgaGTCTTGGAG and which specifically inhibits the expression of GATA-3.

The graph from FIG. 1 shows the viscosity of an hgd40-containing composition. Plotted along the Y-axis of the graph from FIG. 1 is the viscosity of the composition in mPa·s; the X-axis indicates the DNAzyme concentration of the composition. From what is depicted, it emerges that the viscosity of the composition increases essentially exponentially. In this connection, what can be observed especially from a concentration of 50 mg/ml and higher is a sharp rise in viscosity. A viscosity of 3.5 mPa·s can already be recorded at a concentration of about 75 mg/ml.

FIG. 2 shows viscosity measurement results for a DNAzyme-containing composition according to its concentration. They clarify the same relationship between concentration and viscosity as the graph from FIG. 2. Thus, the viscosity increases exponentially in the case of a DNAzyme concentration between 50 mg/ml and 100 mg/ml. This correlation generally cannot be observed to such a pronounced extent for other DNA molecules; usually, the viscosity of a DNA-containing solution rises essentially linearly with rising DNA concentration.

In the case of a viscosity of over 3.5 mPa·s, the pressure which a common inhaler would have to exert for atomization of the composition is too high. This can lead to the inhalers usually used for the treatment of corresponding patients blocking up or clogging up after just a short. This risk is significantly minimized by a composition, the DNAzyme concentration of which is lower than 75 mg/ml.

FIG. 3 depicts measurement results for a FAT of compositions having different DNAzyme concentrations in table form. An FAT (Factory Acceptance Test) is comparable with a factory acceptance test in which the usability of a product is to be tested. The table provides evidence that a DNAzyme concentration of over 75 mg/ml in the composition according to the invention can no longer be atomized by the inhalers from the test (second row of results in the table from FIG. 3). If a composition having a DNAzyme concentration of 75 mg/ml is sterile-filtered before the test run, the concentration is lowered to under 75 mg/ml. A composition having such a DNAzyme concentration can be atomized with the atomizers used in the test, as can be gathered from the fourth and fifth row of results in the table from FIG. 3. The FAT reports the viscosity in dP/ml. Said viscosity should be under 5% to pass the FAT carried out here. The abbreviation “WFI” in the first and third row stands for “Water for Injection” and is to be understood as a control—accordingly, the “WFI” controls correspond to a viscosity of 1 dP/ml.

FIG. 4 shows measurement results for an FAT of the composition according to the invention having DNAzyme concentrations of 20 mg/ml or 50 mg/ml. The results provide evidence that such compositions pass the FAT (Status of FAT test) and can be atomized by means of common inhalers (“Passed” in the rows of results).

The measurement results from FIGS. 3 and 4 thus provide evidence that the composition according to the invention having a DNAzyme concentration of under 75 mg/ml is ideally suited for treating a patient suffering from a respiratory tract disease associated with chronic inflammations because such a composition can be atomized and thus used as an aerosol. In this connection, preference is given to using DNAzyme concentrations of from 20 mg/ml to 50 mg/ml.

It is consequently apparent that the invention relates to a composition for treating a patient suffering from a respiratory tract disease associated with chronic inflammations, the composition comprising at least one DNAzyme which specifically downregulates the expression of GATA-3 and/or comprising at least one DNAzyme which specifically downregulates the expression of Tbet. The composition is characterized in that the concentration of the DNAzyme in the composition is lower than 75 mg/ml, the concentration of the DNAzyme in the composition being preferably between 20 mg/ml and 50 mg/ml.

The invention also relates to a method for producing the composition according to the invention, which composition comprises at least one DNAzyme which specifically downregulates the expression of GATA-3 and/or comprises at least one DNAzyme which specifically downregulates the expression of Tbet. Here too, what is provided is that the concentration of the DNAzyme in the composition is lower than 75 mg/ml, and is preferably lower than 3.5 mPa·s.

The use of the composition is directed to a drug which is used for treating a patient suffering from a respiratory tract disease associated with chronic inflammations.

1. A composition for treating a patient suffering from a respiratory tract disease associated with chronic inflammations, the composition comprising at least one DNAzyme which specifically downregulates the expression of GATA-3 and/or comprising at least one DNAzyme which specifically downregulates the expression of Tbet, characterized in that the concentration of the DNAzyme in the composition is lower than 75 mg/ml.

2. The composition as claimed in claim 1, characterized in that the concentration of the DNAzyme in the composition is between 20 mg/ml and 50 mg/ml.

3. The composition as claimed in claim 1, characterized in that the composition has a viscosity lower than 3.5 mPa·s.

4. The composition as claimed in claim 1, characterized in that the DNAzyme is selected from a group comprising the DNAzymes hgd1 to hgd70 (SEQ ID No. 1 to SEQ ID No. 70) and/or from a group comprising the DNAzymes td1 to td78 (SEQ ID No. 71 to SEQ ID No. 148).

5. The composition as claimed in claim 4, characterized in that the DNAzyme has the sequence hgd40 GTGGATGGAggctagctacaacgaGTCTTGGAG (SEQ ID No. 40).

6. The composition as claimed in claim 1, characterized in that the composition comprises at least one nuclease inhibitor.

7. The composition as claimed in claim 6, characterized in that the nuclease inhibitor specifically inactivates deoxyribonucleases.

8. The composition as claimed in claim 1, characterized in that the composition comprises at least one salt and/or at least one cation.

9. The composition as claimed in claim 1, characterized in that the composition comprises at least one inorganic and/or organic additive and/or a solubilizer and/or a preservative.

10. A method for producing the composition as claimed in claim 1, characterized in that the method comprises at least the following steps:
a) preparing a DNAzyme-containing solution, the DNAzyme concentration in the solution not exceeding 80 mg/ml;
b) filtering the solution from step a) until the solution has a DNAzyme concentration of under 75 mg/ml.

11. The method as claimed in claim 10, characterized in that the method also comprises at least one of the following steps:
adding the at least one nuclease inhibitor to the solution from step b);
adding the at least one salt and/or the at least one cation to the solution from step b);
adding the at least one inorganic and/or organic additive to the solution from step b);
adding the solubilizer and/or the preservative to the solution from step b).

12. A method for treating a patient suffering from a respiratory tract disease associated with chronic inflammations, the method comprising administering a therapeutically effective amount of the composition of claim 1.

13. The method as claimed in claim 12, characterized in that the composition is administered in the form of an aerosol.
